(19) United States
(12) Patent Application Publication
(10) Pub. No.: US 2016/0160164 A1
(43) Pub. Date: Jun. 9, 2016

(54) ANNULAR CULTURE DISH

(71) Applicant: UTI Limited Partnership, Calgary (CA)

(72) Inventors: Mohammed Mehdi Salek, Calgary (CA); Robert J. Martinuzzi, Calgary (CA)

(73) Assignee: UTI Limited Partnership, Calgary, AB (CA)

(21) Appl. No.: 14/907,529

(22) PCT Filed: Jul. 24, 2014
(86) PCT No.: PCT/CA2014/050703

§ 371 Date: Jan. 25, 2016

Related U.S. Application Data
(60) Provisional application No. 61/859,588, filed on Jul. 29, 2013.

(51) Int. Cl. C12M 1/22; C12M 1/12; C12M 1/32

(52) U.S. Cl. CPC C12M 23/10; C12M 23/12; C12M 37/04

(57) ABSTRACT

An annular culture dish comprising a bottom dish component and a top cover lid. The bottom dish component comprises a bottom plate, a continuous sidewall extending upward from the outer perimeter of the bottom plate, and a central column extending upward from the bottom plate about the centre axis of the bottom dish component, said bottom plate, said sidewall and said central column defining an annular chamber.

### TECHNICAL FIELD

Various embodiments disclosed herein generally relate to culture dishes. More specifically, this disclosure pertains to culture dishes defining therein one or more annular culture chambers.

### BACKGROUND

Bacterial biofilms are a significant concern in health care, industrial and environmental processes. Biofilms are difficult to remove from surfaces. On the other hand, the morphologically complex structure of biofilms plays an important role in reducing their susceptibility to antibiotics and host immune systems, and therefore poses serious concerns in different industries where biofilm formation needs to be minimized, especially in medicine. According to National Institutes of Health, 80% of all infections are caused by biofilms. Because of their resistance to antibiotics, biofilms are much more difficult to treat when compared to conventional infections which leads to persistent and recurrent device-associated infections, deterioration of patient life quality, and often replacement of the device. Furthermore, biofilms are responsible for billions of dollars yearly in equipment damage, energy losses, and water system contamination. Development and behavior of biofilms strongly depend on specific local hydrodynamic conditions (e.g. shear stresses) surrounding them. The hydrodynamic mechanisms underlying the formation of biofilms need to be well understood in order to control, prevent and treatment of these “super bugs” as well as to advance the basic understanding of biofilm microbiology.

Recent studies have shown that changes in growth, morphology and susceptibility of biofilms are correlated strongly with different hydrodynamic conditions. A variety of models and assays have been previously developed for in vitro studies of biofilm formation, behavior and susceptibility under different environmental conditions. A major, ubiquitous clinical challenge is conducting many independent tests in parallel within a short time. Some of these tests are used to identify bacterial strains and their phenotypic properties under different flow conditions. A large number of microbial tests are usually conducted to identify an effective antibiotic concentration for a specific type of biofilm, which may require several hundred independent tests before the infection develops beyond control, usually within 24 hours. In spite of the fact that the hydrodynamics is well understood in parallel plate flow chambers, tube flow cells or micro-channels, their use is generally impractical when many concurrent microbial tests over a range of flow conditions are required. The setup for one individual test is expensive, time consuming and usually needs special equipments. It is difficult to keep the replicates identical and they usually have problems such as sealing or manufacturing.

Different types of high-throughput devices can be used for rapid production of biofilms and running multiple tests in parallel. The challenge is then to mimic the desired hydrodynamic conditions in a closed system. Some of these devices are discussed and analyzed in Salek et al. (2010, Numerical simulation of fluid flow and hydrodynamic analysis in commonly used biomedical devices in biofilm studies, IN Numerical Simulations—Examples and Applications in Computational Fluid Dynamics, Lutz Angermann (Ed.), InTech, Chapter 10, pp. 193-212) and in Salek et al. (2011, Analysis of fluid flow and wall shear stress patterns inside partially-filled agitated culture well plates. ABME, DOI: 10. 1007/s10439-011-0444-9). The main advantage of these high-throughput devices is having a repeating geometry with many identical wells. When the plate is translated by an orbital shaker, it provides the same flow condition in all of the wells. While the current high-throughput devices are convenient from a practical perspective, they still have some fundamental disadvantages particularly as related to producing controlled conditions reliably. The complex hydrodynamics arising in these devices usually lead to non-uniform flow conditions along the device such that results are difficult to interpret over the domain with a uniform pulsatile wave shape and relate to specific condition. The shear stress distribution is difficult to control. For example, the temporal or spatial distribution of wall shear stress at different locations of the culture area may make desired conditions only over small portion of the well surface, while deviations significantly are observed across the remaining area of the surface.

### SUMMARY

This section provides a general summary of the disclosure and is not a comprehensive disclosure of its full scope.

The present disclosure pertains to annular culture dishes having annular chambers for continuous flows of culture media therethrough. The continuous flow may comprise a homogenous steady flow, or alternatively a regular pulsating flow, or alternatively an irregular pulsating flow. There are many benefits in using of this new design when comparing to flow cells or existing high-throughput devices. Setup preparation is very fast and does not need any special equipment. The device can generate pulsatile flow very similar to physiological flow without pump-flow cell-tubing setups and problems associated with them in terms of manufacturing and sealing. It is very fast to do replicates in parallel and easy to keep them identical. The shear pulse over the culture areas is directly controlled by the shaker and generally, the rates of fluid flow within each culture well are better controlled when compared to existing high-throughput devices. The device is cheap and disposable and has easy access for optical measurements, microscopy and other analysis. The basic geometry of this device has been designed and optimized using Computational Fluid Dynamics (CFD) analysis.

### BRIEF DESCRIPTION OF THE FIGURES

The drawings described herein are for illustrative purposes only of selected embodiments and are not intended to limit the scope of the present disclosure.

FIG. 1 is a perspective view of an exemplary annular culture dish according to one embodiment of the present invention;

FIG. 2 is a perspective view of the annular channel from the annular culture dish shown in FIG. 1;

FIGS. 3(A) and 3(B) are perspective and side views, respectively, of a fluid's free surface flow within the annular culture plate, at an arbitrary time point;

FIG. 4(A) is a schematic illustration showing the surface shear stress vectors on the bottom face of the annular culture plate from FIGS. 3(A) and 3(B) at the same arbitrary time point, while FIG. 4(B) shows the contours of the magnitude of wall shear stress (Pa) on the bottom face of the annular culture plate (the curved arrow indicates direction of rotation for both FIGS. 3(A) and 3(B));

FIG. 5 is a chart showing the fluctuating shear stresses at the bottom surface of the exemplary annular culture dish during one rotational cycle;

FIG. 6 is a chart showing a pulsatile flow of fluid through three rotational cycles;

FIG. 7 is a chart showing the amplitudes of wall shear stress through three adjacent annular chambers within a single annular culture dish on a gyratory shaker; and

FIG. 8 is a chart showing wall shear stress in each of three annular culture dishes, each dish comprising a single annular chamber and having a different frequency, wherein: (i) the width and diameter of the annular chamber in the first annular culture dish corresponds with the outermost annular chamber in the culture dish from FIG. 7, (ii) the width and diameter of the annular chamber in the second annular culture dish corresponds with the middle annular chamber in the culture dish from FIG. 8, and (iii) the width and diameter of the annular chamber in the third annular culture dish corresponds with the innermost annular chamber in the culture dish from FIG. 7.

### DETAILED DESCRIPTION

The present disclosure pertains to circular culture dishes wherein each culture dish defines one or more symmetrical annular chambers about the centre axis of the dish. Each culture dish may comprise one or more annular chambers. The annular chambers may be concentric annular chambers or alternatively, non-concentric annular chambers. The annular chambers may have different geometries, for example circular, elliptical and the like.

According to one exemplary embodiment of the present disclosure, suitable culture dish approximates the dimensions of a standard Petri dish, wherein the bottom plate component has approximate dimensions of 100 mm diameter with a height of about 10 mm to about 50 mm, about 15 mm to about 40 mm, about 15 mm to about 30 mm, about 15 mm to about 20 mm. The bottom plate is provided with a centre column having a diameter of about 80 mm and the same height as the exterior wall of the bottom plate thereby forming 10-mm wide annular chamber about the periphery of the bottom plate. The top plate contacts the exterior wall of the bottom plate and the top of the centre column, with the outer wall of the top plate extending downward about the outer wall of the bottom plate. A suitable width for the annular chamber defined by the inner wall of the 100-mm diameter bottom plate and the outer diameter of the centre column is about 5 mm, about 10 mm, about 15 mm, about 20 mm, about 25 mm, about 30 mm, about 35 mm, about 40 mm, and therebetween. If so desired, the culture dishes may have a bottom plate with an outer diameter of about 50 mm defining an annular chamber with a width of about 5 mm, about 10 mm, about 15 mm, about 20 mm, and therebetween. If so desired, the culture dishes may have a bottom plate with an outer diameter of about 60 mm defining an annular chamber with a width of about 5 mm, about 10 mm, about 15 mm, about 20 mm, and therebetween. If so desired, the culture dishes may have a bottom plate with an outer diameter of about 70 mm defining an annular chamber with a width of about 5 mm, about 10 mm, about 15 mm, about 20 mm, and therebetween. If so desired, the culture dishes may have a bottom plate with an outer diameter of about 80 mm defining an annular chamber with a width of about 5 mm, about 10 mm, about 15 mm, about 20 mm, about 25 mm, about 30 mm, and therebetween. If so desired, the culture dishes may have a bottom plate with an outer diameter of about 90 mm defining an annular chamber with a width of about 5 mm, about 10 mm, about 15 mm, about 20 mm, about 25 mm, about 30 mm, and therebetween. If so desired, the culture dishes may have a bottom plate with an outer diameter of about 110 mm defining an annular chamber with a width of about 5 mm, about 10 mm, about 15 mm, about 20 mm, about 25 mm, about 30 mm, about 35 mm, about 40 mm, and therebetween. If so desired, the culture dishes may have a bottom plate with an outer diameter of about 120 mm defining an annular chamber with a width of about 5 mm, about 10 mm, about 15 mm, about 20 mm, about 25 mm, about 30 mm, about 35 mm, about 40 mm, and therebetween. If so desired, the culture dishes may have a bottom plate with an outer diameter of about 130 mm defining an annular chamber with a width of about 5 mm, about 10 mm, about 15 mm, about 20 mm, about 25 mm, about 30 mm, about 35 mm, about 40 mm, and therebetween. If so desired, the culture dishes may have a bottom plate with an outer diameter of about 140 mm defining an annular chamber with a width of about 5 mm, about 10 mm, about 15 mm, about 20 mm, about 25 mm, about 30 mm, about 35 mm, about 40 mm, and therebetween. If so desired, the culture dishes may have a bottom plate with an outer diameter of about 150 mm defining an annular chamber with a width of about 5 mm, about 10 mm, about 15 mm, about 20 mm, about 25 mm, about 30 mm, about 35 mm, about 40 mm, and therebetween. If so desired, the culture dishes may have a bottom plate with an outer diameter of about 160 mm defining an annular chamber with a width of about 5 mm, about 10 mm, about 15 mm, about 20 mm, about 25 mm, about 30 mm, about 35 mm, about 40 mm, and therebetween. If so desired, the culture dishes may have a bottom plate with an outer diameter of about 170 mm defining an annular chamber with a width of about 5 mm, about 10 mm, about 15 mm, about 20 mm, about 25 mm, about 30 mm, about 35 mm, about 40 mm, and therebetween. If so desired, the culture dishes may have a bottom plate with an outer diameter of about 180 mm defining an annular chamber with a width of about 5 mm, about 10 mm, about 15 mm, about 20 mm, about 25 mm, about 30 mm, about 35 mm, about 40 mm, and therebetween. Particularly suitable are culture dishes defining an annular chamber have a width selected from a range of about 5 mm to about 20 mm, and a height selected from a range of about 10 mm to about 40 mm. It is to be noted that the centre column may be solid cylinder extending upward from the bottom plate. Alternatively, the centre column may be a ring of material extending upward from the bottom plate.

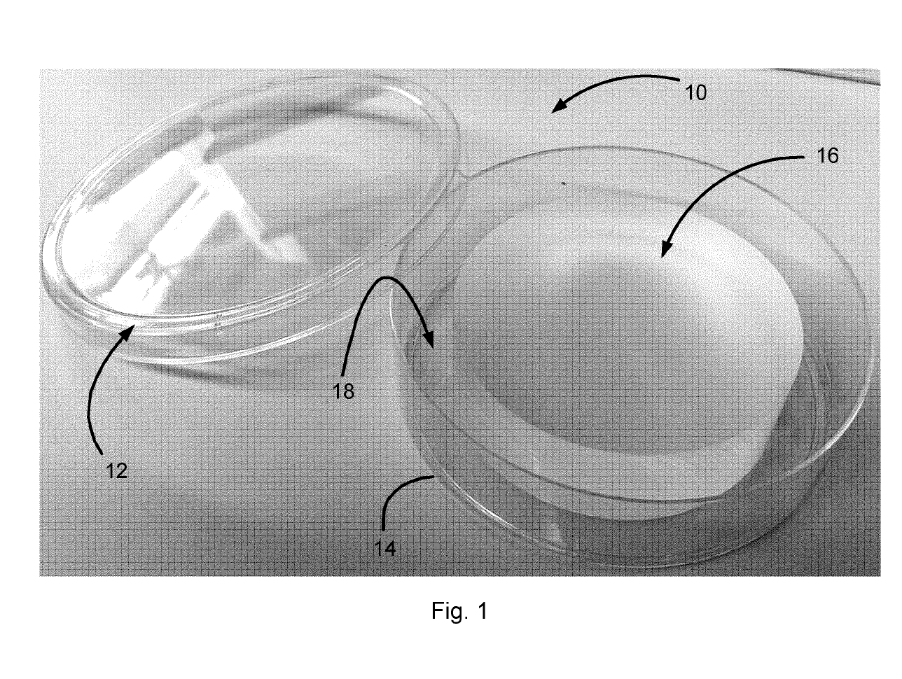

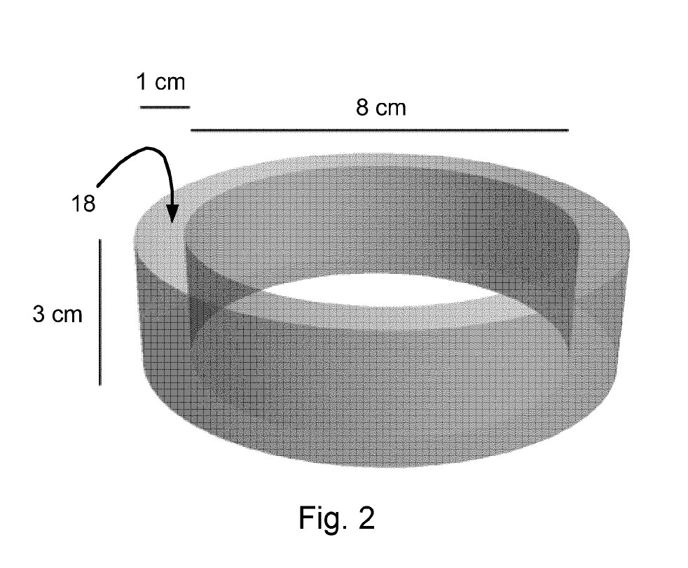

An exemplary annular culture dish is shown in FIGS. 1 and 2. The annular culture dish comprises a standard top plate 12 (a.k.a. a cover plate) and a bottom plate 14. A centre column 16 is integrally engaged with the bottom plate thereby defining an annular chamber 18.

According to another exemplary embodiment of the present disclosure, the bottom plate of the annular culture dish may have two or more annular chambers adjacently disposed about the centre column. If two annular chambers are provided, the width of each chamber may be about 5 mm, about 10 mm, about 15 mm, about 20 mm, and therebetween. If three annular chambers are provided, the width of each chamber may be about 5 mm, about 10 mm, about 15 mm, and therebetween. If four annular chambers are provided, the width of each chamber may be about 5 mm, about 10 mm, about 15 mm, about 20 mm, about 25 mm, about 30 mm, about 35 mm, about 40 mm, about 45 mm, about 50 mm, and therebetween. The two or more annular chambers may be concentric or alternatively, non-concentric.

According to another exemplary embodiment of the present disclosure, a plurality of annular culture wells may be provided within a single square plate. Each annular culture well comprises an circular chamber having an integral centre column thereby providing an annular chamber having a width of about 5 mm, about 7.5 mm, about 10 mm, about 12.5 mm, about 15 mm, and therebetween. A square bottom plate may have four annular culture wells arranged in a 2×2 format, alternatively, nine annular culture wells arranged in a 3×3 format, alternatively, sixteen annular culture wells arranged in a 4×4 format. Alternatively, a square bottom plate may have six annular culture wells arranged such that the centre of each annular culture well is equally spaced from the centre axis of the bottom plate and also, equally spaced from its adjacent annular culture wells. Alternatively, a square bottom plate may have eight annular culture wells arranged such that the centre of each annular culture well is equally spaced from the centre axis of the bottom plate and also, equally spaced from its adjacent annular culture wells.

According to another exemplary embodiment of the present disclosure, a plurality of annular culture wells may be provided within a single rectangular plate. Each annular culture well comprises an circular chamber having an integral centre column thereby providing an annular chamber having a width of about 5 mm, about 7.5 mm, about 10 mm, about 12.5 mm, about 15 mm, and therebetween. A rectangular bottom plate may have, for example, six annular culture wells arranged in a 2×3 format, alternatively eight annular culture wells arranged in a 2×4 format, alternatively twelve annular culture wells arranged in a 3×4 format, or more.

The exemplary annular culture dishes disclosed herein are particularly suitable for culturing cells in liquid cultures. Generally, in applications with culture and well plates, the fluid is adjusted to maintain a fixed fluid depth over the cell cultures. This ensures that, for comparison purposes, the influence of diffusion through the free-surface (exposed to atmosphere) is thus unchanged between multiple experiments. Typical depths of fluids used in experiments are 2 mm and 4 mm above the culture surface. Incubation of cell cultures in annular culture dishes placed onto a rotary shaker provides a constant flow of liquid culture around the annular chamber. When compared to cell culture development in culture dishes that do not have annular chambers, cells cultured in the present annular culture dishes are subject to the same precisely controllable flow conditions throughout an annular chamber which results in significantly reduced variations in the fluid flow environment, minimized cell-to-cell morphological variations, reduced mechanical stresses caused by uneven shear forces typically encountered in prior art culture dishes. The exemplary annular culture dishes are particularly suitable for studies of microbial biofilm formation and manipulation, mammalian cell culture, plant cell culture, protein expression and/or gene expression in mammalian cells and plant cells.

The exemplary annular culture dishes disclosed herein are also suitable for modeling fluid flow dynamics, for example for simulating fluid flow in biological systems exemplified by vascular systems, mammalian cell cultures, gene expression studies and the like, or alternatively for use corrosion testing of materials in difference types of fluids, or alternatively for testing characteristics and properties of coatings. Placing an exemplary annular culture dish onto a gyratory shaker and then modulating the three-dimensional motion around the central axis of the gyratory shaker to provide one of: (i) a regularly reciprocating flow of fluid around the annular chamber, (ii) a regular pulsatile flow of fluid around the annular chamber, and (iii) a flow comprising irregular random pulses. Additionally, fluid flow rates within the annular chambers can be precisely modulated by adjusting the speed of motion of the gyratory shaker.

In order to generate a continuous flow without using bulky pump-flow cell-tubing setups, an appropriate power to move the flow and a continuous geometry are required. One of the available sources of power is the gravity. For example, a periodic height difference can be applied on a continuous geometry to generate a continuous flow controlled by the height difference.

By looking at movement of available shakers (orbital and gyratory shakers), the gyratory shaker seems able to provide a periodic height difference, because the other shaker (orbital shaker) has an in-plane movement. To this aim first the motion of the shaker is analyzed.

In this section, the motion of the gyratory shaker is analyzed to show that it can be controlled for developing a new device.

The gyratory shaker is controlled by an inclination angle of βₛₕₐₖₑᵣ and rotational speed of Ω. Once selected, a complex movement is generated which can be linearly decomposed into three independent movements.

Thus the position of a point in the local coordinate system (X, Y, Z) on the gyratory plate in the absolute frame of reference system can be obtained as a function of time as:

$$[ \begin{matrix}X \\ Y \\ Z\end{matrix} ] = [ \begin{matrix}x {cos}^{2} {α}_{ang} + x cos {β}_{shaker} {sin}^{2} {α}_{ang} - \\ y cos {α}_{ang} sin {α}_{ang} + y cos {β}_{shaker} cos {α}_{ang} sin {α}_{ang} - \\ x sin {α}_{ang} cos {α}_{ang} + x cos {α}_{ang} cos {β}_{shaker} sin {α}_{ang} + \\ y {sin}^{2} {α}_{ang} + y {cos}^{2} {α}_{ang} cos {β}_{shaker} \\ x sin {β}_{shaker} sin {α}_{ang} + y sin {β}_{shaker} cos {α}_{ang}\end{matrix} ] \qquad Eq . 1$$

wherein α_{ang}=Ωt.

The absolute location in the Z-direction provides a relative height difference for a local point on the plate (this height difference results in the gravitational force). The equation for the absolute location in the Z-direction seems able to provide a periodic height difference;

Z=sin βₛₕₐₖₑᵣ(x sin α_{ang}+y cos α_{ang}) Eq. 2

If the absolute location (in the Z-direction) of the points along the desired geometry changes periodically in a way corresponding to their location on the plate, then we have a periodic height difference on this geometry. A symmetric geometry (e.g circle) centered on the plate is able to satisfy this condition.

To this aim, we consider two arbitrary points (x′, y′) and (x″, y″) on a circle centered on the plate and with the radius equal to r′ using Eq. 3:

r′=\sqrt{x′²+y′²} Eq. 3

The Z location of point (x′, y′) is solved using Eq. 4:

Δt=γ₂/Ω Eq. 4

and is equal to the Z location of point (x″, y″) at the current time:

Z_{x′,y′,t}_{₀}_{+Δt}=sin βₛₕₐₖₑᵣ(x sin(γ₁+γ₂)+y cos(γ₁+γ₂))=r′ sin βₛₕₐₖₑᵣ(cos γ₁ sin(γ₁+γ₂)+sin γ₁ cos(γ₁ +γ₂)) Eq. 5

Z_{x″,y″,t}_{₀}=sin βₛₕₐₖₑᵣ(x′ sin(γ₁)+y′ cos(γ₁))=r′ sin βₛₕₐₖₑᵣ(cos(γ₁+γ₂)sin(γ₁)+sin(γ₁+γ₂)cos(γ₁)) Eq. 6

Z_{x′,y′,t}_{₀}_{+Δt}=Z_{x″,y″,t}_{₀} Eq. 7

Therefore, if a continuous geometry (like a torus) is located on the same location of the circle superimposed onto a plate positioned on the shaker table, a continuous flow of a solution driven by gravity within the plate can be provided.

An additional constraint for a cell culture flow chamber is obtained for considering the needs for data collection. Undisturbed optical access is needed for observation and data collection (e.g. microscopy, fluorescence or optical density tests) for which flat surfaces are ideal. To satisfy these constraints, a new design geometry for an annular culture plate according to one of the exemplary embodiments of the present invention is shown in FIGS. 1 and 2.

### Simulation of Flow and Hydrodynamic Analysis of a Solution Flow Patterns in an Annular Culture Plate:

Another embodiment pertains to methods and processes useful for numerical modelling of fluid flow patterns within the exemplary annular culture dish of the present disclosure, in response to complex three-dimensional rotational motions exerted onto the culture dish, using as a starting point the disclosure of Salek at al. (2011, Analysis of Fluid Flow and Wall Shear Stress Patterns Inside Partially-Filled Agitated Culture Well Plates, BMES 40:707-728) for numerical modeling of fluid flow patterns within: (i) a conventional round culture dish, and (ii) a six-well culture dish containing six identical round wells, in response to a two-dimensional rotational force applied by an orbital shaker.

The Salek et al. (2011) numerical model simulating fluid flow dyamincs in response to two-dimensional rotation forces, was simulated using the finite volume-based, commercial Computational Fluid Dynamics (CFD) software FLUENT® flow modelling simulation software (FLUENT is a registered trademark of Ansys Inc., Canonsburg, Pa., USA). Two rotational speeds of the orbital shaker, i.e. 100 rpm and 200 rpm, and two liquid volumes, i.e. 2 ml and 4 ml (four cases in total) were simulated. The free surface motion in the liquid-air interface, the flow patterns inside the well and the mean and instantaneous wall shear stresses were analyzed. Salek et al. (2011) used six-well culture plates, each consisting consists of six identical wells Each well was 18 mm deep and had a radius of R=17.5 mm. A six-well culture plate was mounted onto an orbital shaker and underwent two-dimensional linear translations in the horizontal plane (i.e. XZ plane). The radius of gyration was R_{g}=9.5 mm. Water properties were used to model the liquid phase and air was used to model the gas phase. All properties were at 20° C. and 1 atm: ρ=998.2 kg/m³, μ=0.001003 Pa·s. Each well was filled with 2 ml or 4 ml of liquid medium, corresponding to approximately 2 mm and 4 mm liquid height when at rest. The Reynolds number was defined as

Re=ρΩR_{g}²/μ Eq. 8

following Dardik et al. (2005, Differential effects of orbital and laminar shear stress on endothelial cells. J. Vasc. Surg 41(5): 869-880). The maximum Re used by Salek et al. (2011) as 1886 in order to maintain fluid flow in the laminar regime.

Their two-phase flow was simulated by dividing the solution domain into three regions with two regions corresponding to the homogeneous single-phase fluids (air and water) and one region corresponding to the two-phase water-air interface. The three-dimensional unsteady mass and momentum conservation equations in the homogeneous domain were written, respectively, as:

$$\begin{matrix}\frac{\partial ρ}{\partial t} + ∇ . ( ρ \vec{v} ) = 0 & Eq . 9 \\ \frac{\partial }{\partial t} ( ρ \vec{v} ) + ∇ . ( ρ \vec{v} \vec{v} ) = - ∇ p + ∇ ( μ ∇ \vec{v} ) + ρ \vec{F} & Eq . 10\end{matrix}$$

where \vec{v}, ρ, μ, p and \vec{F} are velocity, density, dynamic viscosity, pressure and external force (per unit mass) for the corresponding single-phase, respectively.

To capture the free surface flow, the volume of fluid (VOF) method was used as laid out in the FLUENT® 6.3 Manual, so that the three domains could be combined into a continuous domain for the solution procedure to ensure that the dynamic condition (shear stress at the free-surface is equal for both phases) was satisfied. Salek et al. (2011) assumed that each fluid phase domain was simply connected. In each computational finite volume at the interface, the volume fraction for each phase, αᵢ, was introduced satisfying:

$${\sum }_{i = 1}^{n} {α}_{i} = 1 \qquad Eq . 11$$

where i represents air or water (i.e. n=2). The continuity and momentum equations are then solved using a modified definition for the fluid properties:

$$ψ = {\sum }_{i = air , \\ water} {α}_{i} {ψ}_{i} \qquad Eq . 12$$

where ψ can be the density or the dynamic viscosity. The system of equations is then closed by satisfying:

$$\frac{\partial {α}_{i}}{\partial t} + \vec{v} . ∇ {α}_{i} = 0 \qquad Eq . 13$$

The geometric Reconstruct Scheme which is the most accurate interface tracking technique (see the FLUENT® 6.3 Manual), was employed for the calculation of the transient VOF model based on the Piecewise-linear approach that assumes that the interface between the two phases has a linear slope in each cell (Youngs, D. L., 1982, Time-Dependent Multi-Material Flow with Large Fluid Distortion. IN Numerical Methods for Fluid Dynamics, K. W. Morton and M. J. Baines (Ed.), Academic Press, New York, N.Y.). Salek et al. (2011) assumed that the influence of surface tension (σ=0.072 N/m for water-air interface at 20° C.) is negligible. This assumption is valid if the gravitational forces and inertial forces on the liquid phase, expressed through the Bond number:

$$( Bo = \frac{( {ρ}_{water} - {ρ}_{air} ) {g ( 2 R )}^{2}}{σ} ) \qquad Eq . 14$$

the Webber number:

$$( We = \frac{{ρ}_{water} {U}^{2} ( 2 R )}{σ} ) \qquad Eq . 15$$

significantly larger than the capillary forces (i.e. Bo, We>>1). Salek et al (2011) determined that in their study, Bo and We were typically of the order of 100.

Salek et al (2011) assumed that an orbital shaker imparts the same two-dimensional, in-plane movement to all points on a six-well culture plate, and therefore, that the velocity of the plate walls was:

$$\vec{U} = [ \begin{matrix}{U}_{x} \\ {U}_{y} \\ {U}_{z}\end{matrix} ] = [ \begin{matrix}- {R}_{g} ΩsinΩ t \\ 0 \\ - {R}_{g} ΩcosΩ t\end{matrix} ] \qquad Eq . 16$$

where R_{g}=9.5 mm was the orbital radius of agitation and Ω was its rotational speed. The solution domain enclosed both fluid phases and extended to the upper lip of the well, although only the results in the liquid layer were presented. A no-slip boundary condition was imposed at the solid walls of the six-well container (i.e. no relative velocity between the fluid and the solid surfaces). The equations of motion were solved in both a stationary and in a moving frame of reference for comparison and validation. For the stationary reference frame, the Dynamic Mesh technique was used, where the entire mesh moves with the velocity imposed by the orbital shaker, \vec{U}. The motion of the moving well was defined using an external C++ user defined function (UDF) linked to FLUENT®. In this frame of reference, the force vector was:

\vec{F}=\vec{g} Eq. 17

The relative frame of reference was non-inertial and translated with the speed imposed by the orbital shaker such that the walls had zero-velocity relative to the frame. The influence of the plate motion was introduced through additional (pseudo-) force terms such that the force vector can be stated as:

$$\vec{F} = \vec{g} - \frac{d \vec{U}}{d t} - \frac{d \vec{ω}}{d t} \times {\vec{R}}_{p} - 2 \vec{ω} \times {\vec{v}}_{rel} - \vec{ω} \times ( \vec{ω} \times {\vec{R}}_{p} ) \qquad Eq . 18$$

where \vec{R}ₚ was the position vector from the origin in the moving frame and ω is the angular velocity of the rotation about the vertical axis of the reference frame. d\vec{U}/dt, d\vec{ω}/dt, 2\vec{ω}×\vec{v}ᵣₑₗ and \vec{ω}×(\vec{ω}×\vec{R}ₚ) were the acceleration of the moving reference frame, the angular acceleration effect, Coriolis and centripetal acceleration, respectively. In orbital motion \vec{ω}=0 and the last three terms in \vec{F} vanish.

Salek et al. (2011) also performed simulations for a single round culture dish and generated numerical grids were generated in GAMBIT® (GAMBIT is a registered trademark of Fluent Inc., Lebanon, N.H., USA). The grids generated in this geometry made it possible to refine the grids near the solid boundaries optimally: 90% of the total grids had a skewness of less than 0.2 and none of the cells had high skewness. The resolution of grids was increased at the walls with a geometric expansion coefficient of 1.04. Grid sensitivity was assessed by nominally doubling the grid density in successive simulations. The wall shear stress was found to be the most sensitive parameter to the change of grid density. The changes in shear stress were negligible between grids of 64,000 and 121,500 cells using dynamic mesh techniques.

Accordingly, numerical simulations were developed for fluid flow patterns within the exemplary annular culture dish of the present disclosure, in response to complex three-dimensional rotational motions exerted onto the culture dish as exemplified by a gyratory shaker by defining a new boundary condition, as explained below.

The volume of fluid (VOF) method was used to capture the free surface flow. On the solid boundaries, a no-slip condition was imposed requiring that there was no relative velocity between the fluid and wall.

In this example, the exemplary annular culture dish is 30 mm deep and has an internal radius of R=40 mm and an external radius of R=50 mm. The culture dish is centered on a gyratory shaker and undergoes a three-dimensional complex motion. The rotational speed is set to 100 rpm and the inclination angle of the plate is set to 6°. The properties of water are used to model the liquid phase and air to model the gas phase. All properties are at 20° C. and 1 atm. 28 ml of water are added to the exemplary annular culture plate which corresponds to a liquid height of approximately 10 mm liquid when the annular culture plate is at rest.

The gyratory shaker imparts a complex three-dimensional movement to all points on within the annular culture plate. When the annular culture plate is located at the center of the plate, the motion of fluid contained within the annular culture plate can be considered a periodic solid body rotation around x and y axis as given by:

$${\vec{ω}}_{ang} = [ \begin{matrix}{ω}_{{ang}_{x}} \\ {ω}_{{ang}_{y}} \\ {ω}_{{ang}_{z}}\end{matrix} ] = [ \begin{matrix}- {β}_{shaker} ΩsinΩ t \\ - {β}_{shaker} ΩcosΩ t \\ 0\end{matrix} ] {θ}_{0} = ( - {β}_{shaker} , 0 , 0 ) \qquad Eq . 8$$

wherein βₛₕₐₖₑᵣ is the inclination angle of the plate, and Ω is its rotational speed.

The equations of motion were solved in a stationary frame of reference and for this purpose the Dynamic Mesh technique was used, where the entire mesh moves with the motion imposed by the gyratory shaker. The motion of the moving annular culture plate was defined using an external C++ user defined function (UDF) linked to the FLUENT® flow modelling simulation software.

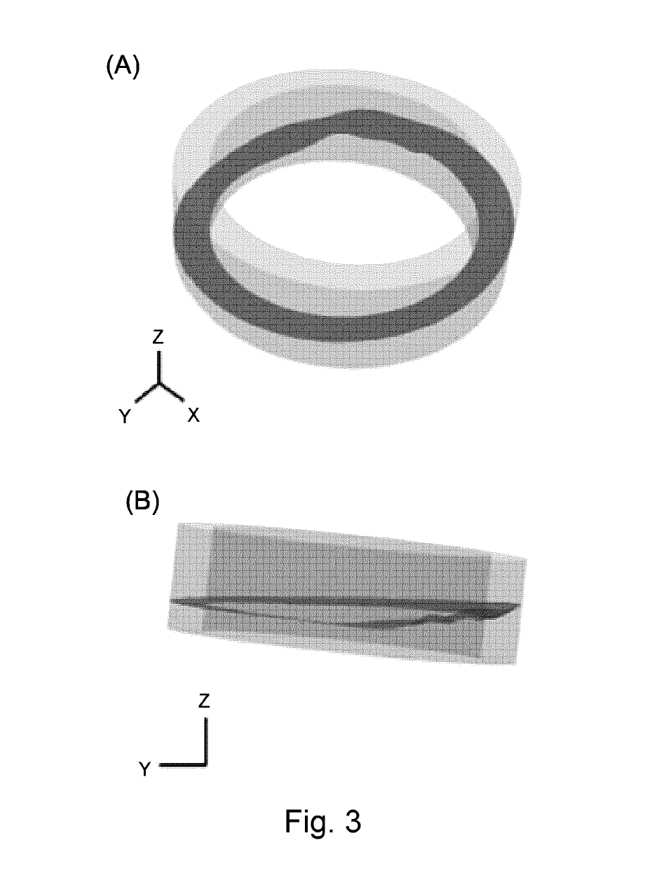

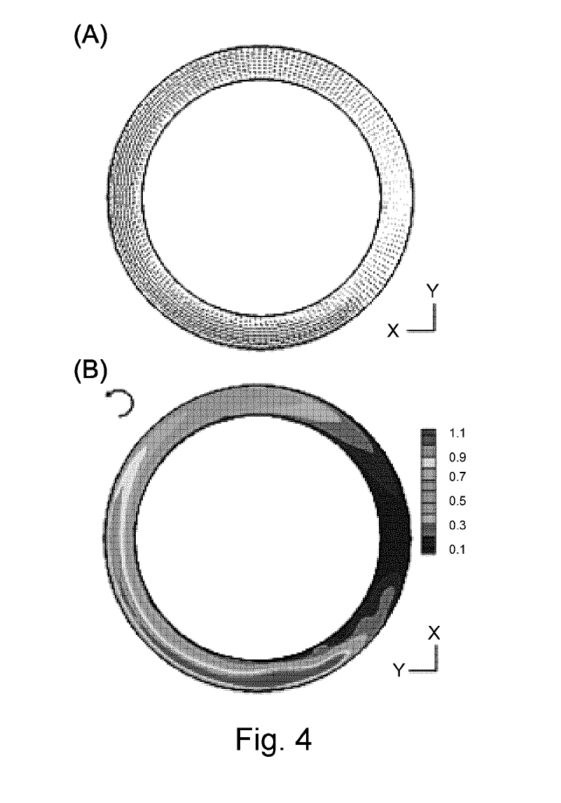

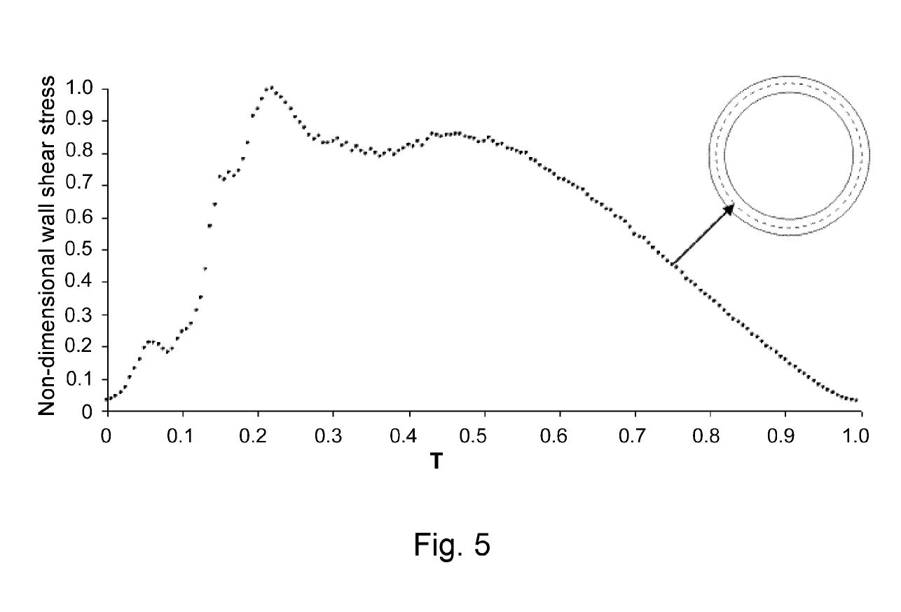

FIGS. 3(A) and 3(B) are perspective and side views, respectively, of a fluid's free surface flow within the annular culture plate, at an arbitrary time point. The free surface is characterized by a travelling wave undergoing solid body rotation at the rate of Ω about the centre axis of the annular culture plate. Like the motion of the free surface, the entire flow field undergoes a solid body rotation about the annular culture plate centre axis. FIG. 4(A) shows the surface shear stress vectors on the bottom face of the annular culture plate at an arbitrary time point during a single rotation cycle. Concurrently, FIG. 4(B) shows the contours of the magnitude of wall shear stress (Pa) on the bottom face of the annular culture plate (the curved arrow indicates direction of rotation for both FIGS. 5(A) and 5(B)). The shear stress field rotates counter-clockwise about the annular culture plate centre axis in the direction and at the rotational rate of the gyratory shaker.

The shear stress vectors are oriented in the direction of the flow on the bottom surface. The shear vectors are mainly uniform (far from the side walls due to no slip boundary conditions) and follow nearly the curvature of the circle. The solid body rotation of the shear stress field, makes a cyclical fluctuation of wall shear stress level for any position on the bottom surface with a period corresponding to the shaker rotation frequency. Consequently, the mean flow and wall stress fields are axisymetric, but the instantaneous flow field contains significant tangential gradients.

FIG. 5 is a graphical representation of the fluctuating shear stresses at the bottom surface of the exemplary annular culture plate during one rotational cycle. The topology of the fluid flow structure here is much simpler when compared to the flow in a moving six-well plate. The flow is mainly aligned tangential to the curvature of each cross section more like the flow in a cross section of a straight channel. The bottom surface of the well is never exposed to air. The flow is accelerated by the gyratory motion of the annular culture plate and gravity and is directed from low liquid level to high fluid elevation along the bottom surface. The wall shear stress increases tangentially and reaches its maximum value under the elevated region (higher fluid level). These flow structures will result in a cyclical fluctuating shear stress when observed at a fixed point as shown in FIG. 5. At this specific rotational speed, there is no flow reversal which may happen at a lower rpm. In this case study, the shaker was set at the maximum angle. Reducing the angle of inclination can lead to more uniform shear stress distribution.

It is to be noted that the data shown in FIGS. 3-5 where based on an exemplary annular culture dish that is 30 mm deep and has an internal radius of R=40 mm and an external radius of R=50 mm, to which is added 28 ml of water. This volume of water corresponds to a liquid height of approximately 10 mm liquid when the annular culture plate is at rest. Suitable exemplary annular dishes may have other dimensions for the inner radius and outer radius of the annular chamber and different volumes of fluid may also be used. For example, an exemplary annular dish may have an outer diameter of 80 mm while the inner radius and outer radius of the annular chamber could be 30 mm and 40 mm respectively. Accordingly, about 4.4 ml of fluid would provide a depth of 2 mm in the annular chamber, while about 8.8 ml of fluid would provide a depth of 4 mm in the annular chamber. Another exemplary annular dish may have an outer diameter of 60 mm while the inner radius and outer radius of the annular chamber could be 20 mm and 30 mm respectively. Accordingly, about 3.1 ml of fluid would provide a depth of 2 mm in the annular chamber, while about 6.3 ml of fluid would provide a depth of 4 mm in the annular chamber.

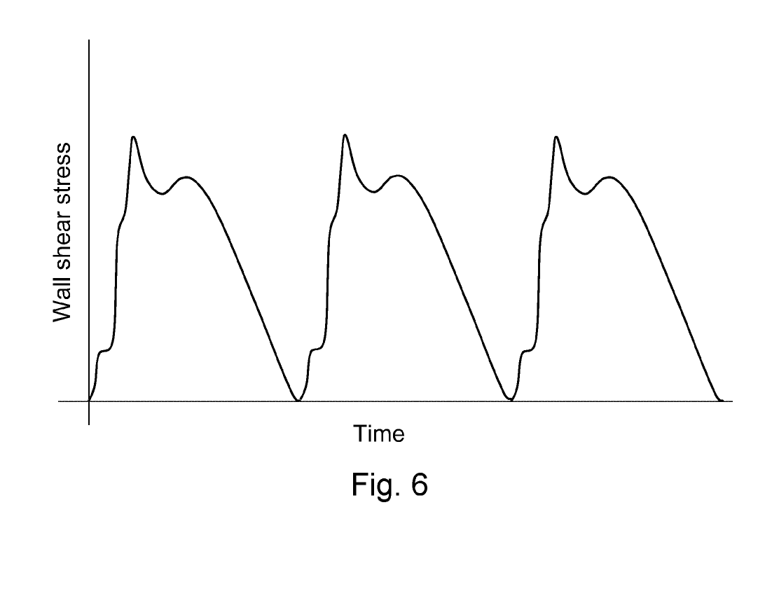

FIG. 6 is a chart showing wall shear stress profiles of a fluid flowing through an annular chamber such as that shown in FIGS. 1 and 2, through three rotational cycles. It is to be noted that for a given gyratory

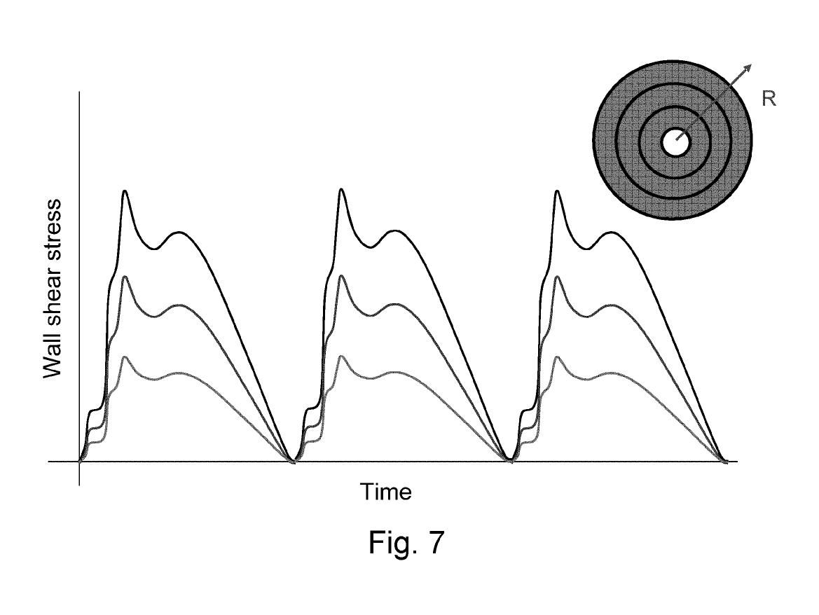

FIG. 7 is a chart showing the amplitudes of wall shear stresses through three adjacent annular chambers within a single annular culture dish on a gyratory shaker. It is to be noted that for a given gyratory frequency, the period (i.e., cycle duration or the reciprocal of frequency) in each channel will be the same, but the amplitude of the wall shear stress will increase as the radius increases. Accordingly, the shortest amplitude was recorded in the annular chamber closest to the central axis of the annular culture dish and the largest amplitude was recorded in the outermost annular chamber.

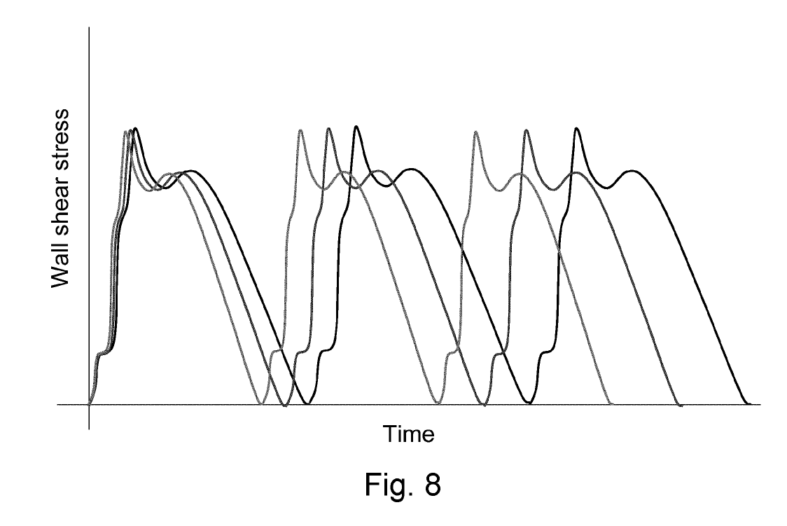

FIG. 8 is a chart showing wall shear stress profiles of fluid flow through three annular culture dishes. Each dish comprises a single annular chamber wherein having a different width from the annular chambers in the other two dishes. Each annular culture dish received the same volume of fluid and was placed on a separate gyratory shaker. The amplitudes of the pulsatile flow in the three annular culture dishes were controlled by separately modulating the speed of each of the three gyratory shakers to provide and maintain approximately equivalent amounts of wall shear stress in the three annular culture dishes. However, the annular culture dish with the smallest-diameter annular chamber has a faster cycle time (red line) than the culture plate with the middle-diameter annular chamber (blue line), which in turn has a faster cycle time than the culture plate with the largest-diameter annular chamber (black line).

1. An annular culture dish comprising:
a bottom dish component defining an annular chamber having a first continuous circular sidewall extending upward from the of the bottom plate and a second continuous circular sidewall extending upward from the bottom plate wherein the diameter of the first continuous circular sidewall is greater than the diameter of the second continuous sidewall; and
a top cover lid for concurrently contacting the tops of said first continuous circular sidewall and said second continuous circular sidewall.

2. An annular culture dish according to claim 1, wherein the second continuous circular sidewall is defined by a solid cylinder of material extending upward from the bottomplate.

3. An annular culture dish according to claim 1, wherein the second continuous circular sidewall is defined by a hollow cylinder of material extending upward from the bottomplate.

4. An annular culture dish according to claim 1, wherein the bottom dish component comprises two or more adjacent annular chambers.

5. An annular dish according to claim 1, wherein the bottom dish component comprises two or more concentric annular chambers.

6. A multi-well culture dish comprising:
a bottom dish component comprising at least two wells, wherein each well is provided with a central column about the centre axis of the well, wherein the inner side surface of the well and the outer side surface of the central column define an annular chamber; and
a top cover lid for contacting the top of said bottom dish component.
